(19) United States
(12) Patent Application Publication
(10) Pub. No.: US 2014/0037760 A1
(43) Pub. Date: Feb. 6, 2014

(54) METHODS OF INDUCING STASIS FOR THE TREATMENT OF CANCER

(71) Applicant: Jeff Thramann, Niwot, CO (US)

(72) Inventor: Jeff Thramann, Niwot, CO (US)

(21) Appl. No.: 14/005,799

(22) PCT Filed: Mar. 19, 2012
(86) PCT No.: PCT/US12/29660

§ 371 Date: Oct. 11, 2013

Related U.S. Application Data
(60) Provisional application No. 61/453,755, filed on Mar. 17, 2011.
(60) Provisional application No. 61/509,519, filed on Jul. 19, 2011.

(51) Int. Cl. A61K 33/04; A61K 31/19

(52) U.S. Cl. CPC A61K 33/04; A61K 31/19
USPC 424/696; 424/708; 424/706; 424/682; 424/702; 424/600; 514/557

(57) ABSTRACT

The present invention relates to methods of treating cancer through stasis induction, wherein said stasis-inducing treatment results in reduced blood flow to the cells of said cancer, leading to diminished tumor growth and/or tumor death. In one embodiment, the stasis-inducing treatment comprises administering a therapeutically effective amount of a chalcogenide, salt, or prodrug thereof. The method of treating cancer may optionally comprise an additional step of withdrawing a portion of blood from said patient, such that blood flow to said cancer is further reduced or eliminated. In additional embodiments, the method of treating cancer may optionally comprise an additional step of diverting a portion of the blood flow in said patient, such that blood flow to said cancer is further reduced or eliminated.

### CROSS-REFERENCE TO RELATED APPLICATIONS

This application claims priority to U.S. Provisional Application Ser. No. 61/453,755, filed Mar. 17, 2011, and U.S. Provisional Application Ser. No. 61/509,519, filed Jul. 19, 2011, both of which are herein incorporated by reference in their entireties.

### TECHNICAL FIELD

The present invention is generally related to methods of treating cancer by subjecting the patient in need thereof to one or more stasis-inducing treatments.

### BACKGROUND

Cancer is a common cause of death in the world. According to the American Cancer Society, over 7 million deaths were attributed to cancer in 2004, making it the second leading cause of death worldwide. Each year, an additional 12 million cases of cancer are diagnosed worldwide, and by 2030, the global burden is expected to grow to 21.4 million new cancer cases and 13.2 million cancer deaths per year, due in large part to population growth and aging of the population. Global Cancer Facts & Figures, 2d ed., 2008.

Currently, the primary modalities of cancer treatment are surgery, chemotherapy, and radiotherapy. There is increasing emphasis worldwide on the development of specialized cancer centers that apply evidence-based multimodal therapies and provide rehabilitation and palliative care. Despite the growing number of cancer therapies, there is still a significant need in the art for novel treatment methods that are useful for treating cancer and other neoplastic diseases without significant side effects, which are frequently seen with currently used chemotherapeutic, surgical, and radiation-based treatments.

The present invention addresses this need through the development of a novel method of cancer treatment which reduces blood flow to tumors and capitalizes on the tumor's inability to regulate itself into a stasis-induced environment and adapt to changing oxygen and nutrient conditions when exposed to conditions that cause the organism to regulate into a stasis-induced environment.

### SUMMARY OF THE INVENTION

The present invention relates to methods of treating cancer through stasis-induction, wherein said stasis-inducing treatment results in reduced blood flow to the cells of said cancer, leading to diminished tumor growth and/or tumor death.

In a first aspect, the present invention relates to a method of treating cancer in a patient in need thereof, comprising subjecting the patient to a stasis-inducing treatment, such that blood flow to said cancer is reduced. In each of the various embodiments described herein, the method of treating cancer may optionally comprise an additional step of withdrawing a portion of blood from said patient, such that blood flow to said cancer is further reduced or eliminated. In additional embodiments, the method of treating cancer may optionally comprise an additional step of diverting a portion of the blood flow in said patient, such that blood flow to said cancer is further reduced or eliminated.

In one embodiment, the stasis-inducing treatment comprises administering a therapeutically effective amount of a chalcogenide, salt, or prodrug thereof. The chalcogenide may be any chalcogenide compound described herein, such as a compound of formula (I) or (IV). In various embodiments described herein, the chalcogenide may comprise sulfur. In one embodiment, the chalcogenide may be sulfide. In another embodiment, the chalcogenide may be a sulfide salt, such as sodium sulfide (Na₂S), sodium hydrogen sulfide (NaHS), potassium sulfide (K₂S), potassium hydrogen sulfide (KHS), lithium sulfide (Li₂S), rubidium sulfide (Rb₂S), cesium sulfide (Cs₂S), ammonium sulfide ((NH₄)₂S), ammonium hydrogen sulfide (NH₄)HS, beryllium sulfide (BeS), magnesium sulfide (MgS), calcium sulfide (CaS), strontium sulfide (SrS), or barium sulfide (BaS). In exemplary embodiments, the chalcogenide may be H₂S, H₂Se, H₂Te or H₂Po.

The chalcogenide compounds of the present invention may be administered intravenously, intradermally, intraarterially, intraperitoneally, intralesionally, intracranially, intraarticularly, intraprostaticaly, intrapleurally, intratracheally, intranasally, intrathecally, intravitreally, intravaginally, intrarectally, topically, intratumorally, intramuscularly, intraperitoneally, intraocularly, subcutaneously, subconjunctival, intravesicularlly, mucosally, intrapericardially, intraumbilically, intraocularally, orally, topically, locally, by inhalation, by injection, by infusion, by continuous infusion, by localized perfusion, via a catheter, or via a lavage.

In various embodiments described herein, the chalcogenide may be provided as a gas, a semi-solid liquid (such as a gel or paste), a liquid, or a solid. It is contemplated that the patient in need thereof may be exposed to more than one such compound and/or to that compound in more than one state. Moreover, the chalcogenide agent may be formulated for a particular mode of administration, as is discussed herein. In certain embodiments, the chalcogenide agent is in pharmaceutically acceptable formulation for intravenous delivery.

In another embodiment, the stasis-inducing treatment comprises exposure of the patient in need thereof to a low oxygen environment. In one embodiment, the patient in need thereof is exposed to an oxygen environment comprising less than about 5% O₂. In another embodiment, the patient in need thereof is exposed to an oxygen environment comprising less than about 4%, less than about 3%, less than about 2%, less than about 1%, less than about 0.5%, less than about 0.1%, less than about 0.01%, or less than about 0.001% O₂. In an exemplary embodiment, the oxygen environment is less than about 0.01% O₂.

In yet another embodiment, the stasis-inducing treatment comprises exposure of the patient in need thereof to a treatment regimen which lowers the core temperature of said patient. In some embodiments, stasis is induced by subjecting the patient to a treatment which reduces the core body temperature by at least about 3° F., 4° F., 5° F., 6° F., 7° F., 8° F., 9° F., or more. In additional embodiments, stasis is induced by subjecting the patient to a treatment which reduces the core body temperature by at least at least about 10° F. (i.e. moderate hypothermia). In further embodiments, stasis is induced by subjecting the patient to a treatment which reduces the core body temperature by at least about 20° F. (i.e. severe hypothermia).

In each of the methods described herein, the patient may additionally be treated with an organ preservation cocktail. The patient may be administered the organ preservation cocktail before, during, and/or after stasis is induced in the patient. In preferred embodiments, the organ preservation cocktail is administered to preserve the function of one or more organs, including, but not limited to, the heart, lungs, kidneys, the liver, the pancreas, and the brain. Administration of the organ preservation cocktail can be by syringe, catheter, pump, or bathing or submersing an organ in a composition comprising the organ preservation cocktail. In one embodiment, the organ preservation cocktail is administered intravenously. In an exemplary embodiment, the organ preservation cocktail is administered via a catheter. In another embodiment, the organ preservation cocktail is cooled prior to administration.

In each of the methods described herein, the patient may additionally be treated with one or more anti-cancer agents. Accordingly, in another aspect, the method of treating cancer comprises the steps of: (a) subjecting the patient to a stasis-inducing treatment, such that blood flow to said cancer is reduced; and (b) administering the patient an anti-cancer agent. The method of treating cancer may optionally comprise an additional step of withdrawing a portion of blood from said patient, such that blood flow to said cancer is further reduced or eliminated. In additional embodiments, the method of treating cancer may optionally comprise an additional step of diverting a portion of the blood flow in said patient, such that blood flow to said cancer is further reduced or eliminated.

In some embodiments, the anti-cancer agent may be selected from an alkylating agent, an antibiotic, an antimetabolic agent, a hormonal agent, a plant-derived agent, or a biologic agent. In specific embodiments, the anti-cancer agent may be selected from dichloroacetate, methotrexate, 6-mercaptopurine, 6-thioguanine, pentostatin, fludarabinphosphate, cladribine, 5-fluorouracil, capecitabine, cytarabin, gemcitabine, hydroxyurea, antinomycin D, daunorubicin, doxorubicin, epirubicin, idarubicin, mitoxantron, bleomycin, mitomycin C, irinotecan, topotecan, mustargen, estramustinphosphate, melphalan, chlorambucil, prednimustine, cyclophosphamide, ifosfamide, trofosfamide, busulfan, treosulfan, thiotepa, carmustin, lomustin, nimustin, dacarbazine, procarbazine, cisplatin, carboplatin, vincristine, vinblastine, vindesine, etoposide, teniposide, paclitaxel, docetaxel, INF-α, prednisone, dexamethasome, G-CSF, aI/-trans retinoic acid, IL-2, GM-CSF, and erythropoietin.

In a specific embodiment, the anti-cancer agent is dichloroacetate or salts thereof or chemical equivalents thereof. Accordingly, in another aspect, the method of treating cancer comprises the steps of: (a) subjecting the patient to a stasis-inducing treatment, such that blood flow to said cancer is reduced; and (b) administering the patient an effective amount of dichloroacetate or salts thereof or chemical equivalents thereof. In a further embodiment of said method, the effective amount of dichloroacetate or salts thereof or chemical equivalents thereof is administered before, during, and/or after the patient is subjected to a statis-inducing treatment. The method of treating cancer may optionally comprise an additional step of withdrawing a portion of blood from said patient, such that blood flow to said cancer is further reduced or eliminated. In additional embodiments, the method of treating cancer may optionally comprise an additional step of diverting a portion of the blood flow in said patient, such that blood flow to said cancer is further reduced or eliminated.

The methods of the present invention are useful in the treatment in a wide variety of cancers, including, but not limited to, solid tumors (e.g., tumors of the lung, breast, colon, prostate, bladder, rectum, brain or endometrium), hematological malignancies (e.g., leukemias, lymphomas, myelomas), carcinomas (e.g., bladder carcinoma, renal carcinoma, breast carcinoma, colorectal carcinoma), neuroblastoma, or melanoma. Non-limiting examples of these cancers include cutaneous T-cell lymphoma (CTCL), noncutaneous peripheral T-cell lymphoma, lymphoma associated with human T-cell lymphotrophic virus (HTLV), adult T-cell leukemia/lymphoma (ATLL), acute lymphocytic leukemia, acute nonlymphocytic leukemia, chronic lymphocytic leukemia, chronic myelogenous leukemia, Hodgkin's disease, non-Hodgkin's lymphoma, multiple myeloma, mesothelioma, childhood solid tumors such as brain neuroblastoma, retinoblastoma, Wilms' tumor, bone cancer and soft-tissue sarcomas, common solid tumors of adults such as head and neck cancers (e.g., oral, laryngeal and esophageal), genito urinary cancers (e.g., prostate, bladder, renal, uterine, ovarian, testicular, rectal and colon), lung cancer, breast cancer, pancreatic cancer, melanoma and other skin cancers, stomach cancer, brain cancer, liver cancer, adrenal cancer, kidney cancer; thyroid cancer, basal cell carcinoma, squamous cell carcinoma of both ulcerating and papillary type, metastatic skin carcinoma, medullary carcinoma, osteo sarcoma, Ewing's sarcoma, veticulum cell sarcoma, Kaposi's sarcoma, neuroblastoma and retinoblastoma.

### DETAILED DESCRIPTION

The present inventor has discovered that stasis-inducing treatments can be used as an effective therapy for cancer. These stasis-inducing treatments can include, but are not limited to, exposure to a chalcogenide, exposure to low oxygen environments, and/or exposure to hypothermic temperatures. Each of these stasis-inducing treatments results in the reduced blood flow to tumor cells. The present inventor has found that unlike normal tissue, tumor cells are unable to execute the complex and highly regulated cell signaling mechanisms required to enter into a protective state when exposed to stressed environments (e.g., environments exposed to stasis-inducing treatments such as a chalcogenide therapy which blocks oxidative phosphorylation). In contrast to tumor cells, normal tissue responds to a stasis-inducing treatment by entering a state of hibernation (i.e., suspended animation), a state which protects the normal tissue from excessive cellular damage. The present inventor has found that through this process, normal tissue is preserved while cancer cells undergo apoptosis. Thus, the present invention capitalizes on the tumor's inability to adapt to changing oxygen and nutrient conditions when exposed to a stasis-induced environment. The present inventor has also found that the anti-tumor effect of the stasis environment can be further augmented by reducing blood flow to the tumor (i.e., through the removal of a portion of the patient's blood, or diverting a portion of the patient's blood flow away from the tumor). Therefore, some methods of the invention are designed to further decrease blood flow to the tumor to enhance the effectiveness of the anti-tumor effects of the stasis-inducing treatments.

Accordingly, one aspect of the present invention is directed to the use of stasis-inducing treatments alone, or optionally with one more additional treatments, such as blood withdrawal, blood diversion, and/or the use of traditional chemotherapeutic agents, for the prevention and/or treatment of cancer, which is a group of diseases characterized by the uncontrolled growth and spread of abnormal cells.

As described herein, the present inventor has discovered that stasis-inducing treatments are effective for the treatment of cancer. As used herein, the term “stasis” is used interchangeably with “suspended animation” to refer to a state in which a cell, tissue, organ, or organism (collectively referred to as “biological material”) is living, but where the cellular functions necessary for cell division, developmental progression, and/or metabolic state are slowed or even stopped. As described in WO/2005/041655 to Roth et al., stasis can be used as a method of preservation by itself, or it may be induced as part of a cryopreservation regimen. Stasis-inducing treatments are known to reduce physical and physiological damage to living tissue and entire organisms by reducing or eliminating stress or physical injury. Stasis may be beneficial by decreasing the need of biological material for oxygen and, therefore, blood flow. Thus, it may extend the period of time that biological material can be isolated from a life-sustaining environment and exposed to a death-inducing environment. Methods for inducing a state of stasis are described in WO/2005/041655, which is herein incorporated by reference in its entirety for all purposes.

To date, the potential utility of stasis-inducing treatments for the treatment of cancer have neither been explored nor characterized. The present inventor has discovered that cancer cells are unable to regulate themselves to slow down their metabolism when exposed to stasis conditions (e.g., if the patient is subjected to a stasis-inducing treatment, or if a portion of the patient's anatomy containing the tumor is subjected to a stasis-inducing treatment). Accordingly, the present inventor describes herein several methods for treating cancer via the induction of a stasis environment. The induction of stasis may result from exposure to a chalcogenide, exposure to a low oxygen environment, and/or exposure to one or more hypothermic treatments. Each of these treatments may be combined with additional therapies to further stress the tumor cells, including decreasing the blood flow to the tumor cells by withdrawing a portion of the blood to the area surrounding the tumor cell, decreasing the blood flow to the tumor cells by diverting a portion of the blood flow in said patient, and/or treatment with traditional chemotherapeutic agents.

The patient may be any patient of a species such as human, non-human primate, canine, feline, porcine, bovine, equine, ovine, and murine which may benefit from the methods of treating cancer described herein. The methods of the invention accordingly find use in the treatment and prevention of cancer in patients in need thereof, as well as the prevention of cancer metastasis in said patients.

### Chalcogenides for Inducing Stasis

In one embodiment, the stasis-inducing treatment comprises administering a therapeutically effective amount of a chalcogenide, a chalcogenide salt, or prodrug thereof to a patient in need thereof. As described herein, any chalcogenide compound can be used, so long as it achieves a goal of the invention (e.g., induction of stasis in a patient or a portion of the patient's anatomy). Exemplary chalcogenides for induction of stasis are described in US 2008/0171725 to Roth et al., which is incorporated herein by reference in its entirety for all purposes.

In certain embodiments, the chalcogenide compound comprises sulfur, selenium, tellurium, or polonium. In certain embodiments, a chalcogenide compound contains one or more exposed sulfide groups. These chalcogenide compounds may contain 1, 2, 3, 4, 5, 6 or more exposed sulfide groups. In particular embodiments, such a sulfide-containing compound is CS₂ (carbon disulfide). In an exemplary embodiment, the chalcogenide is hydrogen sulfide.

In some methods of the invention, stasis is induced in a patient or a portion of the patient's anatomy by exposing the patient or a portion of the patient's anatomy to a chalcogenide that is selected from hydrogen sulfide (H₂S), sodium sulfide (Na₂S), sodium hydrogen sulfide (NaHS), potassium sulfide (K₂S), potassium hydrogen sulfide (KHS), lithium sulfide (Li₂S), rubidium sulfide (Rb₂S), cesium sulfide (Cs₂S), ammonium sulfide ((NH₄)₂S), ammonium hydrogen sulfide (NH₄)HS, beryllium sulfide (BeS), magnesium sulfide (MgS), calcium sulfide (CaS), strontium sulfide (SrS), barium sulfide (BaS), hydrogen selenide (H₂Se), and hydrogen telluride (H₂Te).

In some methods of the invention, stasis is induced in a patient or a portion of the patient's anatomy by exposing the patient or a portion of the patient's anatomy to a reducing agent that has a chemical structure of Formula I:

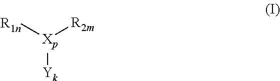

wherein X is N, O, Po, S, Se, or Te;
wherein Y is N or O,
wherein R₁ is H, C, lower alkyl, a lower alcohol, or CN;
wherein R₂ is H, C, lower alkyl, a lower alcohol, or CN;
wherein n is 0 or 1;
wherein m is 0 or 1;
wherein k is 0, 1, 2, 3, or 4; and,
wherein p is 1 or 2.

The terms “lower alkyl” and “lower alcohol” are used according to their ordinary meanings and the symbols are the ones used to refer to chemical elements. This chemical structure will be referred to as the “reducing agent structure” and any compound having this structure will be referred to as a reducing agent structure compound. In additional embodiments, k is 0 in the reducing agent structure. Moreover, in other embodiments, the R₁ and/or R₂ groups can be an amine or lower alkyl amine. In others, R₁ and/or R₂ could be a short chain alcohol or a short chain ketone. Additionally, R₁ and R₂ may be a linear of branched chain bridge and/or the compound may be a cyclic compound. In still further embodiments, X may also be a halogen. The term “lower” is meant to refer to 1, 2, 3, 4, 5, or 6 carbon atoms, or any range derivable therein. Moreover, R₁ and/or R₂ may be other small organic groups, including, C₂-C₅ esters, amides, aldehydes, ketones, carboxylic acids, ethers, nitrites, anhydrides, halides, acyl halides, sulfides, sulfones, sulfonic acids, sulfoxides, and/or thiols. Such substitutions are clearly contemplated with respect to R₁ and/or R₂. In certain other embodiments, R₁ and/or R₂ may be short chain versions of the small organic groups discussed above. “Short chain” means 1, 2, 3, 4, 5, 6, 7, 8, 9, 10, 11, or 12 carbon molecules, or any range derivable therein.

In certain embodiments, the chalcogenide is a salt, preferably salts wherein the chalcogen is in a −2 oxidation state. Sulfide salts encompassed by embodiments of the invention include, but are not limited to, sodium sulfide (Na₂S), sodium hydrogen sulfide (NaHS), potassium sulfide (K₂S), potassium hydrogen sulfide (KHS), lithium sulfide (Li₂S), rubidium sulfide (Rb₂S), cesium sulfide (Cs₂S), ammonium sulfide ((NH₄)₂S), ammonium hydrogen sulfide (NH₄)HS, beryllium sulfide (BeS), magnesium sulfide (MgS), calcium sulfide (CaS), strontium sulfide (SrS), barium sulfide (BaS), and the like. The present invention also encompasses corresponding selenide and telluride salts. In an exemplary embodiment, the chalcogenide is hydrogen sulfide.

It is specifically contemplated that the invention includes compositions containing a chalcogenide salt (i.e., a chalcogenide compound that is a salt) with a pharmaceutically acceptable carrier or prepared as a pharmaceutically acceptable formulation. In still further embodiments, the reducing agent structure compound is selected from the group consisting of H₂S, H₂Se, H₂Te, and H₂Po. In some cases, the reducing agent structure of Formula (I) has an X that is an S. In others, X is Se, or X is Te, or X is Po, or X is O. Furthermore, k in the reducing agent structure is 0 or 1 in some embodiments. In certain embodiments, the reducing agent structure compound is dimethylsulfoxide (DMSO), dimethylsulfide (DMS), carbon monoxide, methylmercaptan (CH₃SH), mercaptoethanol, thiocyanate, hydrogen cyanide, methanethiol (MeSH), or CS₂.

In certain embodiments, a selenium-containing compound such as H₂Se is employed. The amount of H₂Se may be in the range of 1 to 1000 parts per billion in some embodiments of the invention. It is further contemplated that any embodiment discussed in the context of a sulfur-containing compound may be implemented with a selenium-containing compound. This includes substituting one of more sulfur atoms in a sulfur-containing molecule with a corresponding selenium atom.

In some methods of the invention, stasis is induced in a patient or a portion of the patient's anatomy by exposing the patient or a portion of the patient's anatomy to a reducing agent that has a chemical structure of Formula IV:

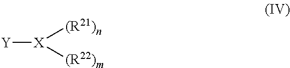

wherein X is N, O, P, Po, S, Se, Te, O—O, Po—Po, S—S, Se—Se, or Te—Te;
wherein n and m are independently 0 or 1;
wherein R²¹ and R²² are independently hydrogen, halo, cyano, phosphate, thio, alkyl, alkenyl, alkynyl, alkoxy, aminoalkyl, cyanoalkyl, hydroxyalkyl, haloalkyl, hydroxyhaloalkyl, alkylsulfonic acid, thiosulfonic acid, alkylthio sulfonic acid, thioalkyl, alkylthio, alkylthioalkyl, alkylaryl, carbonyl, alkylcarbonyl, haloalkylcarbonyl, alkylthiocarbonyl, aminocarbonyl, aminothiocarbonyl, alkylaminothiocarbonyl, haloalkylcarbonyl, alkoxycarbonyl, aminoalkylthio, hydroxyalkylthio, cycloalkyl, cycloalkenyl, aryl, aryloxy, heteroaryloxy, heterocyclyl, heterocyclyloxy, sulfonic acid, sulfonic alkyl ester, thiosulfate, or sulfonamido; and
Y is cyano, isocyano, amino, alkyl amino, aminocarbonyl, aminocarbonyl alkyl, alkylcarbonylamino, amidino, guanidine, hydrazino, hydrazide, hydroxyl, alkoxy, aryloxy, hetroaryloxy, cyloalkyloxy, carbonyloxy, alkylcarbonyloxy, haloakylcarbonyloxy, arylcarbonyloxy, carbonylperoxy, alkylcarbonylperoxy, arylcarbonylperoxy, phosphate, alkylphosphate esters, sulfonic acid, sulfonic alkyl ester, thiosulfate, thiosulfenyl, sulfonamide, —R²³R²⁴, wherein R²³ is S, SS, Po, Po—Po, Se, Se—Se, Te, or Te—Te, and R²⁴ is defined as for R²¹, or Y is

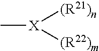

wherein X, R²¹ and R²², are as defined herein.

### Methods of Administering Chalcogenides

As described herein, in one embodiment, the stasis-inducing treatment comprises administering a therapeutically effective amount of a chalcogenide, a chalcogenide salt, or prodrug thereof to a patient in need thereof.

As used herein, a “therapeutically effective amount” means an amount that can achieve the stated result. In certain methods of the invention, a “therapeutically effective amount” is, for example, an amount that induces stasis in the patient in need thereof. In some embodiments, a “therapeutically effective amount” may refer to an amount that increases apoptosis in tumor cells and/or decreases the size of a tumor following administration to a patient in need thereof. The “therapeutically effective amount” can be determined based on a current comparison or a previous comparison to untreated patients or patients treated with a different dosage or regimen that do not experience stasis, and the concomitant increased apoptosis of tumor cells and/or decrease in the size of a tumor.

Methods for measuring stasis are described in US 2008/0171725 to Roth et al., which is incorporated herein by reference in its entirety for all purposes. Briefly, It will be understood that when inducing stasis in a tissue or organ, an effective amount is one that induces stasis in the tissue or organ as determined by the collective amount of cellular respiration of the tissue or organ. Accordingly, for example, if the level of oxygen consumption by a particular tissue (e.g., a pancreas) (collectively with respect to cells of the pancreas) is decreased at least about 2-fold (i.e., 50%) after exposure to a particular amount of a certain stasis-inducing compound, it will be understood that that was an effective amount to induce stasis in the pancreas. Similarly, an effective amount of an agent that induces stasis in a patient is one that is evaluated with respect to the collective or aggregate level of a particular parameter of stasis. It will be also understood that when inducing stasis in a patient, an effective amount is one that induces stasis generally of the whole patient, unless a particular part of the patient was targeted for stasis. In addition, it is understood that an effective amount may be an amount sufficient to induce stasis by itself, or it may be an amount sufficient to induce stasis in combination with another agent or stimuli, e.g., another active compound or exposure to another stasis-inducing treatment such as a low oxygen environment or hypothermic conditions.

In some embodiments, stasis can be measured indirectly by a drop in core body temperature of an organism. It is contemplated that a reduction in core body temperature of about, at least about, or at most about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50° F. or more, or any range derivable therein may be observed in the stasis-inducing methods of the invention.

In accordance with the invention, one or more chalcogenides can be administered to a patient in need thereof via inhalation, injection, catheterization, immersion, lavage, perfusion, topical application, absorption, adsorption, or oral administration. Said chalcogenides may be administered intravenously, intradermally, intraarterially, intraperitoneally, intralesionally, intracranially, intraarticularly, intraprostaticaly, intrapleurally, intratracheally, intranasally, intrathecally, intravitreally, intravaginally, intrarectally, topically, intratumorally, intramuscularly, intraperitoneally, intraocularly, subcutaneously, subconjunctival, intravesicularlly, mucosally, intrapericardially, intraumbilically, intraocularally, orally, topically, locally, by inhalation, by injection, by infusion, by continuous infusion, by localized perfusion, via a catheter, or via a lavage. In the case of an isolated tumor, one of more chalcogenides can be administered locally. Where the tumor has spread to multiple organs, one or more chalcogenides can be administered systemically.

In some embodiments, the pharmaceutical composition is a liquid. In one embodiment, the composition may be a liquid with the relevant compound(s) dissolved or bubbled into the composition. In some embodiments, the chalcogenide formulation further includes a pharmaceutically acceptable diluent. In one embodiment, the chalcogenide formulation comprises a buffering agent. In another embodiment, the chalcogenide is provided in a first sealed container and the pharmaceutically acceptable diluent is provided in a second sealed container. In some embodiments, the chalcogenide comes in a kit comprising instructions for mixing the chalcogenide and the diluent. Additionally, the chalcogenide can be reconstituted for achieving any method of the invention, such as for inducing stasis in a patient in need thereof. It is contemplated that any label would specify the result to be achieved (e.g., increased apoptosis in tumor cells and/or decreased tumor size in conjunction with the treatment of cancer).

In certain embodiments, the pharmaceutical composition contains an effective dose of a chalcogenide to produce a therapeutically effective benefit. In certain embodiments, the Cmaxor steady state plasma concentration to be achieved is about, at least about, or at most about 0.01, 0.1, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 3000, 4000, 5000, 6000, 7000, 8000, 9000, 10000 μM or more, or any range derivable therein.

In other embodiments, the chalcogenide is administered as a gas. In some embodiments, the chalcogenide is in a gas mixture comprising more than one gas. In some embodiments, the other gas is a noble gas (helium, neon, argon, krypton, xenon, radon, or ununoctium), nitrogen, nitrous oxide, hydrogen, or a mixture thereof. In various embodiments described herein, a gaseous chalcogenide may be first diluted with a non-toxic and/or non-reactive gas prior to administration to a patient.

As described herein, the chalcogenide, a chalcogenide salt, or prodrug thereof can be administered to a patient need thereof for the purpose of treating cancer. The method of treating cancer with the chalcogenide may optionally comprise an additional step of reducing blood flow to the tumor (i.e., through the removal of a portion of the patient's blood, or diverting a portion of the patient's blood flow away from the tumor) as described herein.

### Use of Low Oxygen Environments for Inducing Stasis

In another embodiment, the stasis-inducing treatment comprises exposure of the patient in need thereof to a low oxygen environment. In one embodiment, the patient in need thereof is exposed to an oxygen environment comprising less than about 5% O₂. In another embodiment, the patient in need thereof is exposed to an oxygen environment comprising less than about 4%, less than about 3%, less than about 2%, less than about 1%, less than about 0.5%, less than about 0.1%, less than about 0.01%, or less than about 0.001% O₂. In an exemplary embodiment, the oxygen environment is less than about 0.01% O₂.

Accordingly, the present invention takes advantage of the understanding that patients can undergo a state of stasis (i.e., suspended animation) when exposed to hypoxic conditions. In some embodiments, stasis is induced in the patient by exposing the patient in need thereof to hypoxic conditions sufficient to induce stasis of the patient or a portion of the patient's anatomy. It is contemplated that “sufficient to induce stasis” means that the patient or a portion of the patient's anatomy is exhibiting signs of stasis, i.e., for a finite length of time (as opposed to death). Such stasis is characterized by a lack of movement, absence of cell division, reduction in cell division, absence of heartbeat, reduced heart beat, and lack of or reduction in developmental progression as observed by light microscopy.

As discussed in further detail below, hypoxic conditions include conditions in which the oxygen concentration is less than 20.8%—the concentration of normal atmospheric conditions—and as low as 0.01 or 0.001% (near anoxic conditions); thus hypoxic conditions includes anoxic condition. Accordingly, it is contemplated that hypoxic conditions with more than 0% oxygen are part of the invention. In some embodiments, oxygen concentration is less than 20, 19, 18, 17, 16, 15, 14, 13, 12, 11, 10, 9, 8, 7, 6, 5, 4, 3, 2, 1, 0.5, 0.1, 0.01, or 0.001%. It is also contemplated that in most embodiments of the invention, the patient or a portion of the patient's anatomy will be restored to normal atmospheric (i.e., normoxic) conditions, allowing for stasis to be reversed. It is further contemplated that minimal damage or harm to the patient will result from being in stasis under the conditions described herein.

Methods for the induction of stasis in a patient using exposure to low oxygen environments are described in US 2010/0021387 to Roth et al., which is incorporated herein by reference in its entirety for all purposes. Briefly, such methods include first incubating the patient under hypoxic or anoxic conditions for an effective amount of time for the patient to enter stasis and then, optionally, lowering the temperature of the patient. Accordingly, in each of the methods of the claimed invention, the patient may additionally be exposed to temperatures lower than room temperature, including temperatures that will freeze the patient. Lowering of temperature may increase the duration that the patient may undergo reversible stasis and prevent damage or further damage to patient, or increase the efficacy of a stasis inducer such as a chalcogenide or exposure to a low oxygen environment. In some embodiments, the patient may be exposed to hypoxic conditions and placed in a temperature lower than room temperature either to prevent damage to the patient or to prevent further damage to the patient.

As described herein, in addition to hypoxic/anoxic conditions, another way of inducing stasis is to administer an effective amount of a stasis inducer compound (e.g., a chalcogenide), which is a compound capable of inducing a patient to enter stasis, preferably reversible stasis. Therefore, in each of the methods described herein, the patient may be subjected to a combination of treatments designed to induce stasis. In one embodiment, the patient may be administered a chalcogenide and subjected to hypoxic/anoxic conditions. In another embodiment, the patient may be administered a chalcogenide and subjected to hypothermic conditions. In yet another embodiment, the patient may be subjected to hypoxic/anoxic conditions and further subjected to hypothermic conditions. Methods for inducing stasis via hypothermic treatments are described in further detail below.

As described herein, a patient in need thereof may be exposed to a low oxygen environment for the purpose of treating cancer. The method of treating cancer via exposure to a low oxygen environment may optionally comprise an additional step of reducing blood flow to the tumor (i.e., through the removal of a portion of the patient's blood, or diverting a portion of the patient's blood flow away from the tumor) as described herein.

### Use of Hypothermic Treatments for Inducing Stasis

In another embodiment, the stasis-inducing treatment comprises exposure of the patient in need thereof to hypothermic conditions.

As described herein, hypothermia occurs when the body temperature of a patient, or temperature of a body region or organ of the patient, becomes reduced below the normal body temperature in a warm-blooded animal. As used herein, the term “normal body temperature” refers to temperature of about 37° C., that is, preferably between about 35° C. to about 39° C., most preferably between about 36° C. to about 38° C.

Hypothermia has been used for treating a variety of disease conditions in human patients for several years. For example, U.S. Pat. Nos. 4,750,493 and 4,920,963 to Brader are directed to methods for cooling the extracranial area during emergency care of cardiac arrest of severe shock in order to induce vasoconstriction and intracranial hypothermia. These inventions employed topical cold packs and/or watertight shrouds applied to the head. U.S. Pat. No. 5,383,854 to Safar et al. is directed to a cardio-pulmonary bypass apparatus adaptable to include a module that comprises a heat exchanger capable of cooling the blood, while U.S. Pat. No. 5,464,834 to Peglion et al. is directed to chemical compounds acting as a 5-HT₁A receptor, wherein said chemical compounds are capable of inducing hypothermia. Methods and devices for reducing the body temperature of a patient are further described in U.S. Pat. Nos. 7,008,445, 7,507,250 and 7,621,945 to Lennox et al., 7,231,771 to McMurry et al., and 6,962,601 to Becker et al., each of which is incorporated herein by reference in its entirety for all purposes.

Accordingly, hypothermia in human patients may be induced in a variety of ways, including the use of topical cold packs, the administration of chemical compounds including muscle relaxants and sedatives, the administration of iced fluids, and the use of cardio-pulmonary bypass devices equipped with heat exchangers. Additional methods for inducing hypothermia are generally described in US 2002/0138121, which is incorporated herein by reference in its entirety for all purposes. These and other methods for producing hypothermia in patients have the effect of inducing stasis.

In some embodiments, stasis is induced by subjecting the patient to a treatment which reduces the core body temperature by at least about 3° F., 4° F., 5° F., 6° F., 7° F., 8° F., 9° F., or more. In additional embodiments, stasis is induced by subjecting the patient to a treatment which reduces the core body temperature by at least about 10° F. (i.e., moderate hypothermia). In further embodiments, stasis is induced by subjecting the patient to a treatment which reduces the core body temperature by at least about 20° F. (i.e., severe hypothermia).

In one embodiment, the induction of hypothermia is systemic. The induction of systemic hypothermia in a patient has the effect of inducing stasis. In one embodiment, systemic hypothermia is applied by immersion of the patient's body in a cool bath. In another embodiment, systemic hypothermia is applied using a conventional systemic hypothermia system. Such conventional systems include blankets or pads where cooled water is circulated through channels in the walls of the blanket or pad and the patient's body contacts the walls of the blanket.

In another embodiment, systemic hypothermia is induced using a catheter which is inserted into the bloodstream of the patient. For example, U.S. Pat. No. 3,425,419 to Dato describes a cathetar-based method of lowering and raising the temperature of the human body. In one embodiment, hypothermia is induced in a patient using a metallic catheter. The metallic catheter has an inner passageway through which a fluid, such as water, can be circulated. The catheter is inserted through the femoral vein and then through the inferior vena cava as far as the right atrium and the superior vena cava.

In another embodiment, systemic hypothermia is induced in a patient by local cooling the surface of the patient's head. For example, a conventional head-cooling device involves a head cap with a gel substance contained within the walls of the cap. Prior to use, for example, a user (e.g., medical technician) places the head-cooling device in a freezer to reduce the temperature of the gel within the cap. During operation, the user fits the reduced-temperature cap to the head of a patient. The gel within the walls of the cap absorbs heat from the head, thereby cooling the head of the patient.

In additional embodiments, systemic hypothermia may be induced in a patient by providing contact between a tissue region of interest and a cooling fluid. For example, one conventional device finding use in the present invention includes a flexible hood having multiple ribs or studs disposed on the inner surface of the hood. When a user places the hood on a head of a patient, the ribs or studs contact the head and maintain a fluid circulation space between the head and the hood and an edge, defined by the hood, which contacts the patient's skin. A negative pressure source draws a cooling fluid through the flexible hood, under negative pressure, to cause the fluid to contact the scalp of the patient and draw heat away from (e.g., cool) the scalp. Furthermore, application of the negative pressure seals the edges of the hood against the skin of the patient (e.g., a region substantially free of hair).

In other embodiments, the induction of hypothermia is local. Local hypothermia refers to cold being applied to very small areas, such as a tumor or a tissue surrounding a tumor. Local hypothermia in the region of suspected tumor cells may be the preferred method of stasis-induction where the tumor is isolated. In another embodiment, the induction of hypothermia is regional. Regional hypothermia involves treating an organ or limb (e.g., selective organ hypothermia may be employed via perfusion of a cold solution such as saline or perfluorocarbons). Additional treatment protocols for the induction of local and/or regional hypothermia are known in the art and many devices for the application of local and/or regional hypothermia are known. Devices for inducing local hypothermia and/or regional hypothermia are described in U.S. Pat. No. 4,154,245 to Daily, U.S. Pat. No. 6,033,383 to Ginsburg, and U.S. Pat. No. 6,251,130 to Dobak et al., each of which is herein incorporated by reference in its entirety for all purposes.

As described herein, a patient in need thereof may be exposed to hypothermic conditions for the purpose of treating cancer. The method of treating cancer via exposure to hypothermic conditions may optionally comprise an additional step of reducing blood flow to the tumor (i.e., through the removal of a portion of the patient's blood, or diverting a portion of the patient's blood flow away from the tumor) as described herein.

### Use of Organ Preservation Cocktails for Maintaining Organ Function During Stasis Induction

During the induction of stasis, hypoxia can occur. Tissue hypoxia begins a cascade of events in the cells of tissues and organs that quickly leads to damage. During prolonged periods of hypoxia, tissue and organ damage is often irreversible. Accordingly, in each of the methods described herein, the patient who is subjected to a stasis-inducing therapy may additionally be treated with an organ preservation cocktail to prevent or mitigate tissue and organ damage that may result from stasis induction. Organ preservation cocktails can comprise metabolic inhibitors which preserve the function of organs deprived of oxygen for extended periods of time. The present invention provides methods wherein the patient subjected to a stasis-inducing treatment is administered an organ preservation cocktail designed to allow the tissue of an organ to regain proper metabolic activity and function.

Accordingly, the invention provides in one embodiment a method of preserving or maintaining an organ, comprising contacting the organ with the solution for organ preservation or maintenance. The patient may be administered the organ preservation cocktail before, during, and/or after stasis is induced in the patient.

In preferred embodiments, the organ preservation cocktail is administered to preserve the function of one or more organs, including, but not limited to, the heart, lungs, kidneys, the liver, the pancreas, and the brain.

In one embodiment, the organ preservation cocktail is an aqueous solution for organ preservation or maintenance. Examples of aqueous solutions for the preservation of organs are described in U.S. Pat. No. 4,798,824 to Belzer et al., U.S. Pat. No. 5,552,267 to Stern et al., U.S. Pat. No. 6,994,954 to Taylor, U.S. Pat. No. 7,537,885 to Fischer et al., and U.S. Pat. No. 7,718,617 to Cicardi et al., each of which is incorporated herein by reference in its entirety for all purposes. Other formulations for organ preservation include 1) the Stanford University solution [See, e.g., Swanson et al., 1988, Journal of Heart Transplantation, 7: 456-467] 2) a modified Collins solution [See, e.g., Maurer et al., 1990, Transplantation Proceedings 22: 548-550 and 3) the University of Wisconsin solution (Belzer et al., U.S. Pat. No. 4,798,824, issued Jan. 17, 1989).

Generally, the organ preservation formulations are buffered solutions comprising salts, such as calcium chloride, potassium chloride, or magnesium chloride, and substrates such as glutamate or aspartate.

In some embodiments, the organ preservation formulations comprise electrolytes and antioxidants. In one embodiment, the organ preservation formulation is a plasma expander. Plasma expanders suitable for use in the present invention are described in U.S. Pat. No. 4,874,742 to Ecanow et al., U.S. Pat. Nos. 5,130,230, 5,571,801, and 6,627,393 to Segall et al., and U.S. Pat. No. 6,746,836 to Widra, each of which is incorporated herein by reference in its entirety for all purposes. Plasma expanders of the present invention will generally comprise an aqueous solution of electrolytes at physiological concentration, a biological buffer having a buffering capacity in the range of physiological pH, simple nutritive sugar or sugars, and magnesium ions in a concentration sufficient to substitute the flux of calcium across cell membranes.

In some embodiments, the tissue- or organ-preserving formulations can comprise a lipophilic local anesthetic. Preferred lipophilic local anesthetics include, but are not limited to, bupivacaine, levo-bupibacaine, etidocaine, ropivacaine, and tetracaine. The effective amount of the anesthetic in the composition is an amount sufficient to protect a tissue or organ from damage due to acidosis, oxidative damage, ischemia and repurfusion injury during the absence of adequate blood flow. As will be understood by those of skill in the art, the effective amount may differ depending on the desired tissue or organ to protect (e.g., brain, heart, lung, kidney, liver, or pancreas), the method of administering or contacting the tissue or organ with the anesthetic, or the size of the organ.

Administration of the organ preservation cocktail can occur via syringe, catheter, pump, or by bathing or submersing an organ in a composition comprising the organ preservation cocktail. In one embodiment, the organ preservation cocktail is administered intravenously. In an exemplary embodiment, the organ preservation cocktail is administered via a catheter.

In one embodiment, the organ preservation cocktail is administered systemically. In another embodiment, the organ preservation cocktail is administered locally (e.g., in or around the site of localized stasis induction). Systemic or selective administration of the organ preservation cocktail is administered as a means to prolong the window by which stasis may be induced in a patient.

In some embodiments, the organ preservation cocktail can be administered using perfusion devices. A perfusion device as used herein is any mechanical device that can be used to infuse a specific organ or the systemic circulation with a solution comprising the organ preservation cocktail. The device may include a tube, catheter, or cannula leading from the reservoir that can be inserted into an organ, vein, or artery. The device can be an electromechanical device having electric pumps for controlling the temperature, rate or volume of delivery of the solution. Exemplary devices include those commercially available by BARD Inc., and those described in U.S. Pat. Nos. 5,011,469 to Buckberg et al. and 6,221,063 to Barra et al., both of which are incorporated herein by reference in their entireties for all purposes.

### Targeted Tumor Treatment

In various embodiments described herein, the tumor targeted for treatment may be an isolated tumor. When the tumor is an isolated tumor, additional techniques for reducing the blood flow to the isolated tumor may be preferred. Thus, the present invention is useful as a method of treating a tumor having a discrete blood supply, where one or more blood vessels that feed the tumor are blocked, thereby cutting off blood supply to the tumor resulting in reduced growth and/or death of the tumor.

In one embodiment, blood flow is diverted away from the tumor site via the use of a diversion catheter. Such diversion catheters are described in U.S. Pat. No. 7,540,583 to Hayzelden. In one embodiment, the diversion catheter includes a catheter body having a proximal end and a distal end. An inflation lumen is in fluid connection with an inflation mechanism situated proximate the proximal end of the catheter body. A diversion balloon is fixably mounted at the distal end of the catheter body and inflatable to at least partially block blood flow through the vessels near the tumor site. One or more distal ports are provided on the catheter body distal to the diversion balloon. One or more proximal ports are provided on the catheter body proximal to the diversion balloon. The proximal ports are in fluid connection with the distal ports. A flow of blood through the vessels near the tumor site is directed via the distal and proximal ports so as to divert blood flow away from a tumor site targeted for treatment.

### Use of Heart-Lung Bypass Machines for Treatment of Metastasized Cancers

In various embodiments described herein, the cancer targeted for treatment may be a cancer which has metastasized to multiple organs. In some cases, the cancer may reside in multiple organs, but not the brain due to the blood brain barrier. It is well known that periods of reduced blood flow to the brain can seriously diminish or damage brain function. Accordingly, in one embodiment, blood flow may be maintained to the brain through the use of a heart-lung bypass during the induction of stasis and/or removal of blood, thereby preserving blood flow to the brain while decreasing blood flow to the remainder of the body.

### Cancers Suitable for Treatment

The methods of the present invention are useful in the treatment of cancer or neoplastic disorders.

As used herein, the term “cancer” or “neoplastic disorder” means neoplastic growth, hyperplastic growth or proliferative growth or a pathological state of abnormal cellular development and includes solid tumors, non-solid tumors, and any abnormal cellular proliferation, such as that seen in leukemia.

The methods of the present invention are expected to be useful in the treatment of a broad range of cancers, including, but not limited to, non-solid tumors, such as leukemia and multiple myeloma, hematological malignancies such as lymphoma, and also solid tumors and their metastases.

In one embodiment, the cancer or neoplastic disorder targeted for treatment with a method of the present invention is a solid tumor. Solid tumors which may be treatable by the methods of the present invention include head and neck cancers (e.g., oral, laryngeal and esophageal), genito urinary cancers (e.g., prostate, bladder, renal, uterine, ovarian, testicular, rectal and colon), lung cancer, breast cancer, pancreatic cancer, melanoma and other skin cancers, stomach cancer, brain cancer, liver cancer, adrenal cancer, kidney cancer, thyroid cancer, basal cell carcinoma, squamous cell carcinoma of both ulcerating and papillary type, metastatic skin carcinoma, medullary carcinoma, osteo sarcoma, Ewing's sarcoma, veticulum cell sarcoma, Kaposi's sarcoma, neuroblastoma and retinoblastoma.

In additional embodiments, the cancer or neoplastic disorder targeted for treatment with a method of the present invention is a non-solid tumor or hematological malignancy, including, but not limited to, leukemias, lymphomas, and myelomas. Hematological malignancies which may be treatable by the methods of the present invention include cutaneous T-cell lymphoma (CTCL), noncutaneous peripheral T-cell lymphoma, lymphoma associated with human T-cell lymphotrophic virus (HTLV), adult T-cell leukemia/lymphoma (ATLL), acute lymphocytic leukemia, acute nonlymphocytic leukemia, chronic lymphocytic leukemia, chronic myelogenous leukemia, Hodgkin's disease, non-Hodgkin's lymphoma, and multiple myeloma.

Combination with Chemotherapeutic Agents

In each of the methods described herein, the patient may additionally be treated with one or more anti-cancer agents. Accordingly, in another aspect, the method of treating cancer comprises the steps of: (a) subjecting the patient to a stasis-inducing treatment, such that blood flow to said cancer is reduced; and (b) administering the patient an anti-cancer agent. The method of treating cancer may optionally comprise an additional step of withdrawing a portion of blood from said patient, such that blood flow to said cancer is further reduced or eliminated. In additional embodiments, the method of treating cancer may optionally comprise an additional step of diverting a portion of the blood flow in said patient, such that blood flow to said cancer is further reduced or eliminated.

In some embodiments, the anti-cancer agent may be selected from an alkylating agent, an antibiotic, an antimetabolic agent, a hormonal agent, a plant-derived agent, or a biologic agent. In specific embodiments, the anti-cancer agent may be selected from dichloroacetate, methotrexate, 6-mercaptopurine, 6-thioguanine, pentostatin, fludarabinphosphate, cladribine, 5-fluorouracil, capecitabine, cytarabin, gemcitabine, hydroxyurea, antinomycin D, daunorubicin, doxorubicin, epirubicin, idarubicin, mitoxantron, bleomycin, mitomycin C, irinotecan, topotecan, mustargen, estramustinphosphate, melphalan, chlorambucil, prednimustine, cyclophosphamide, ifosfamide, trofosfamide, busulfan, treosulfan, thiotepa, carmustin, lomustin, nimustin, dacarbazine, procarbazine, cisplatin, carboplatin, vincristine, vinblastine, vindesine, etoposide, teniposide, paclitaxel, docetaxel, INF-α, prednisone, dexamethasome, G-CSF, aI/-trans retinoic acid, IL-2, GM-CSF, and erythropoietin.

The treatment methods described herein may be achieved by way of simultaneous, sequential, or separate administration of the individual components of the treatment (e.g., a stasis-inducing treatment followed by treatment with one or more chemotherapeutic agents). In one embodiment, the dosage ranges of these pharmaceutically-active agents will be within the approved dosage ranges, when used in combination treatments employing the methods and compositions of the present invention.

Combination with Dichloroacetate

In certain exemplary embodiments, the stasis-inducing treatment may be administered in conjunction with an effective amount of dichloroacetate or salts thereof or chemical equivalents thereof. Accordingly, in another aspect, the application relates to a method of treating cancer that comprises the steps of: (a) subjecting the patient to a stasis-inducing treatment, such that blood flow to said cancer is reduced; and (b) administering the patient an effective amount of dichloroacetate or salts thereof or chemical equivalents thereof. The method of treating cancer may optionally comprise an additional step of withdrawing a portion of blood from said patient, such that blood flow to said cancer is further reduced or eliminated. In additional embodiments, the method of treating cancer may optionally comprise an additional step of diverting a portion of the blood flow in said patient, such that blood flow to said cancer is further reduced or eliminated.

Compositions comprising dichloroacetate or salts thereof or chemical equivalents thereof and methods of said compositions for the treatment of cancer are described in US 2009/0118370 to Michelakis et al., which is incorporated herein by reference in its entirety for all purposes. Dichloroacetate or salts thereof or chemical equivalents thereof have the effect of reversing apoptosis resistance in cancer cells, such as cancer cells with hyperpolarized mitochondria, such as non-small cell lung cancers, glioblastomas, and breast carcinomas. The present inventor has discovered that the anti-cancer effects of dichloroacetate are enhanced when administered in conjunction with a stasis-inducing treatment. Without being bound to any theory, it is believed that the dichloroacetate has the effect of blocking glycolysis in the mitochondria of cancer cells, forcing them to metabolize via the more efficient and oxygen demanding oxidative phosphorylation process of aerobic metabolism. When dichloroacetate is administered in conjunction with a stasis-inducing treatment, the present inventor has discovered that the combination augments a tumor's inability to adapt to changing oxygen and nutrient conditions, leading to tumor apoptosis.

In one embodiment, the dichloroacetate is a salt of dichloroacetic acid. In another embodiment, the dichloroacetic acid is a sodium salt of dichloroacetic acid.

In some embodiments, the dichloroacetate, a salt thereof, or a chemical equivalent thereof, is administered in the form of a pharmaceutical composition comprising dichloroacetate, a salt thereof, or a chemical equivalent thereof and a pharmaceutically acceptable carrier. In yet another embodiment the invention provides a use of dichloroacetate, a salt thereof, or a chemical equivalent thereof in the preparation of a medicament or pharmaceutical composition for the treatment of cancer. In yet another embodiment, the dichloroacetate, a salt thereof, or a chemical equivalent thereof, is administered orally.

In some embodiments, the dichloroacetate, a salt thereof, or a chemical equivalent thereof is administered in a water-based formulation. In one embodiment the water-based formulation of dichloroacetate, a salt thereof, or a chemical equivalent thereof comprises at least about 0.0075 g/L of dichloroacetate, a salt thereof, or a chemical equivalent thereof. In additional embodiments, the water-based formulation of dichloroacetate, a salt thereof, or a chemical equivalent thereof comprises at least about 0.075 g/L, at least about 0.75 g/L, or at least about 7.5 g/L of dichloroacetate, a salt thereof, or a chemical equivalent thereof). In another embodiment, the dichloroacetate, a salt thereof, or a chemical equivalent thereof is administered at a total daily dose of at least about 10 mg/kg of dichloroacetate, a salt thereof, or a chemical equivalent thereof. In additional embodiments, the dichloroacetate, a salt thereof, or a chemical equivalent thereof is administered at a total daily dose of at least about 20 mg/kg, at least about 25 mg/kg, at least about 30 mg/kg, at least about 35 mg/kg, at least about 40 mg/kg, at least about 45 mg/kg, at least about 50 mg/kg, at least about 60 mg/kg, at least about 70 mg/kg; at least about 80 mg/kg, at least about 90 mg/kg, or at least about 100 mg/kg dichloroacetate, a salt thereof, or a chemical equivalent thereof.

In one embodiment, the effective amount of dichloroacetate or salts thereof or chemical equivalents thereof is administered before the patient is subjected to a statis-inducing treatment. In another embodiment, the effective amount of dichloroacetate or salts thereof or chemical equivalents thereof is administered as the patient is being subjected to a statis-inducing treatment. In yet another embodiment, the effective amount of dichloroacetate or salts thereof or chemical equivalents thereof is administered after the patient has been subjected to a statis-inducing treatment. In yet another embodiment, the effective amount of dichloroacetate or salts thereof or chemical equivalents thereof is administered before, during, and/or after the patient is subjected to a statis-inducing treatment.

As used herein, the phrase “dichloroacetate or salts thereof or chemical equivalents thereof” refers to dichloroacetate acid or salt thereof or other analog, derivative of dichloroacetate that has the same desired therapeutic effect in the treatment of cancer.

Salts of dichloroacetic acid are well known and commercially available. Generally, such salts of dichloroacetic acid will have the following formula:

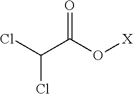

wherein X=Na, K⁺, CH₃ or OH.

Specific salts include those formed by the alkali metal and alkaline earth metal ions such as sodium, potassium, calcium, and magnesium, ammonium, and substituted ammonium where the substituent is a mono-ordi-lower alkyl radical of 1-4 carbon atoms and ethylene diammonium. Exemplary pharmaceutically acceptable salts are those with minimum cell cytotoxicity, such as sodium.

Exemplary pharmaceutical suitable for use in conjunction with a stasis-inducing treatment include sodium dichloroacetate, potassium dichloroacetate, and diisopropyl ammonium dichloroacetate. In one embodiment, salts of dichloroacetic acid can be used at a concentration between about 0.5 mM to about 100 mM. In some embodiments, such compounds are used at a concentration of at concentrations of 0.1-10 mM. In further embodiments, such compounds are used at a concentration at least about 0.5 mM.

The foregoing detailed description has been given for clearness of understanding only and no unnecessary limitations should be understood there from as modifications will be obvious to those skilled in the art.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

The disclosures, including the claims, figures and/or drawings, of each and every patent, patent application, and publication cited herein are hereby incorporated herein by reference in their entireties.

1. A method of treating cancer in a patient in need thereof, comprising subjecting the patient in need thereof to a stasis-inducing treatment.

2. The method of claim 1, wherein said stasis-inducing treatment comprises administering a therapeutically effective amount of a chalcogenide, a chalcogenide salt, or prodrug thereof.

3. The method of claim 2, wherein said chalcogenide is a compound of formula (I): 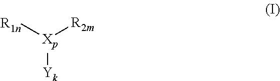
wherein X is N, O, Po, S, Se, or Te;
wherein Y is N or O,
wherein R₁ is H, C, lower alkyl, a lower alcohol, or CN;
wherein R₂ is H, C, lower alkyl, a lower alcohol, or CN;
wherein n is 0 or 1;
wherein m is 0 or 1;
wherein k is 0, 1, 2, 3, or 4; and,
wherein p is 1 or 2.

4. The method of claim 2, wherein said chalcogenide is a compound of formula (IV): 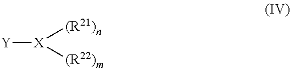
wherein X is N, O, P, Po, S, Se, Te, O—O, Po—Po, S—S, Se—Se, or Te—Te;
wherein n and m are independently 0 or 1;
wherein R²¹ and R²² are independently hydrogen, halo, cyano, phosphate, thio, alkyl, alkenyl, alkynyl, alkoxy, aminoalkyl, cyanoalkyl, hydroxyalkyl, haloalkyl, hydroxyhaloalkyl, alkylsulfonic acid, thiosulfonic acid, alkylthio sulfonic acid, thioalkyl, alkylthio, alkylthioalkyl, alkylaryl, carbonyl, alkylcarbonyl, haloalkylcarbonyl, alkylthiocarbonyl, aminocarbonyl, aminothiocarbonyl, alkylaminothiocarbonyl, haloalkylcarbonyl, alkoxycarbonyl, aminoalkylthio, hydroxyalkylthio, cycloalkyl, cycloalkenyl, aryl, aryloxy, heteroaryloxy, heterocyclyl, heterocyclyloxy, sulfonic acid, sulfonic alkyl ester, thiosulfate, or sulfonamido; and
Y is cyano, isocyano, amino, alkyl amino, aminocarbonyl, aminocarbonyl alkyl, alkylcarbonylamino, amidino, guanidine, hydrazino, hydrazide, hydroxyl, alkoxy, aryloxy, hetroaryloxy, cyloalkyloxy, carbonyloxy, alkylcarbonyloxy, haloakylcarbonyloxy, arylcarbonyloxy, carbonylperoxy, alkylcarbonylperoxy, arylcarbonylperoxy, phosphate, alkylphosphate esters, sulfonic acid, sulfonic alkyl ester, thiosulfate, thiosulfenyl, sulfonamide, —R²³R²⁴ wherein R²³ is S, SS, Po, Po—Po, Se, Se—Se, Te, or Te—Te, and R²⁴ is defined as for R²¹, or Y is 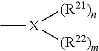
wherein X, R²¹ and R²², are as defined herein.

5. The method of claim 2, wherein said chalcogenide is selected from hydrogen sulfide (H₂S), sodium sulfide (Na₂S), sodium hydrogen sulfide (NaHS), potassium sulfide (K₂S), potassium hydrogen sulfide (KHS), lithium sulfide (Li₂S), rubidium sulfide (Rb₂S), cesium sulfide (Cs₂S), ammonium sulfide ((NH₄)₂S), ammonium hydrogen sulfide (NH₄)HS, beryllium sulfide (BeS), magnesium sulfide (MgS), calcium sulfide (CaS), strontium sulfide (SrS), barium sulfide (BaS), hydrogen selenide (H₂Se), and hydrogen telluride (H₂Te).

6. The method of claim 2, wherein said chalcogenide is administered intravenously, intradermally, intraarterially, intraperitoneally, intralesionally, intracranially, intraarticularly, intraprostaticaly, intrapleurally, intratracheally, intranasally, intrathecally, intravitreally, intravaginally, intrarectally, topically, intratumorally, intramuscularly, intraperitoneally, intraocularly, subcutaneously, subconjunctival, intravesicularlly, mucosally, intrapericardially, intraumbilically, intraocularally, orally, topically, locally, by inhalation, by injection, by infusion, by continuous infusion, by localized perfusion, via a catheter, or via a lavage.

7. The method of claim 2, wherein said chalcogenide is provided as a gas, semi-solid liquid, liquid, or solid.

8. The method of claim 1, wherein said stasis-inducing treatment comprises exposure of the patient in need thereof to a low oxygen environment.

9. The method of claim 8, wherein said patient in need thereof is exposed to an oxygen environment comprising less than about 1% O₂, less than about 0.1% O₂, less than about 0.01% O₂, or about 0.001% O₂.

10-12. (canceled)

13. The method of claim 1, wherein said stasis-inducing treatment comprises exposure of the patient in need thereof to a treatment regimen which lowers the core temperature of said patient.

14. The method of claim 13, wherein the core body temperature of the patient is reduced by at least about 5° F., by at least about 10° F., or by at least about 20° F.

15. (canceled)

16. (canceled)

17. The method of claim 1, wherein a portion of blood from said patient is withdrawn, such that blood flow to said cancer is reduced or eliminated.

18. The method of claim 1, wherein a portion of blood in said patient is diverted away from said cancer, such that blood flow to said cancer is reduced or eliminated.

19. The method of claim 1, wherein said patient in need thereof is additionally administered an anti-cancer agent.

20. The method of claim 19, wherein said anti-cancer agent is selected from an alkylating agent, an antibiotic, an antimetabolic agent, a hormonal agent, a plant-derived agent, or a biologic agent.

21. The method of claim 19, wherein said anti-cancer agent is selected from methotrexate, 6-mercaptopurine, 6-thioguanine, pentostatin, fludarabinphosphate, cladribine, 5-fluorouracil, capecitabine, cytarabin, gemcitabine, hydroxyurea, antinomycin D, daunorubicin, doxorubicin, epirubicin, idarubicin, mitoxantron, bleomycin, mitomycin C, irinotecan, topotecan, mustargen, estramustinphosphate, melphalan, chlorambucil, prednimustine, cyclophosphamide, ifosfamide, trofosfamide, busulfan, treosulfan, thiotepa, carmustin, lomustin, nimustin, dacarbazine, procarbazine, cisplatin, carboplatin, vincristine, vinblastine, vindesine, etoposide, teniposide, paclitaxel, docetaxel, INF-α, prednisone, dexamethasome, G-CSF, aI/-trans retinoic acid, IL-2, GM-CSF, and erythropoietin.

22. The method of claim 19, wherein said anti-cancer agent is dichloroacetate or salts thereof or chemical equivalents thereof.

23. A method of treating cancer comprising the steps of: (a) subjecting the patient to a stasis-inducing treatment; and (b) administering to the patient an effective amount of dichloroacetate or salts thereof or chemical equivalents thereof.

24. The method of claim 23, wherein said stasis-inducing treatment comprises administering a therapeutically effective amount of a chalcogenide, a chalcogenide salt, or prodrug thereof.

25. The method of claim 24, wherein said chalcogenide is a compound of formula (I): 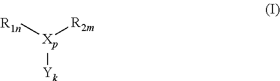
wherein X is N, O, Po, S, Se, or Te;
wherein Y is N or O,
wherein R₁ is H, C, lower alkyl, a lower alcohol, or CN;
wherein R₂ is H, C, lower alkyl, a lower alcohol, or CN;
wherein n is 0 or 1;
wherein m is 0 or 1;
wherein k is 0, 1, 2, 3, or 4; and,
wherein p is 1 or 2.

26. The method of claim 24, wherein said chalcogenide is a compound of formula (IV): 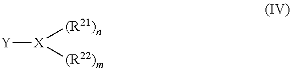
wherein X is N, O, P, Po, S, Se, Te, O—O, Po—Po, S—S, Se—Se, or Te—Te;
wherein n and m are independently 0 or 1;
wherein R²¹ and R²² are independently hydrogen, halo, cyano, phosphate, thio, alkyl, alkenyl, alkynyl, alkoxy, aminoalkyl, cyanoalkyl, hydroxyalkyl, haloalkyl, hydroxyhaloalkyl, alkylsulfonic acid, thiosulfonic acid, alkylthio sulfonic acid, thioalkyl, alkylthio, alkylthioalkyl, alkylaryl, carbonyl, alkylcarbonyl, haloalkylcarbonyl, alkylthiocarbonyl, aminocarbonyl, aminothiocarbonyl, alkylaminothiocarbonyl, haloalkylcarbonyl, alkoxycarbonyl, aminoalkylthio, hydroxyalkylthio, cycloalkyl, cycloalkenyl, aryl, aryloxy, heteroaryloxy, heterocyclyl, heterocyclyloxy, sulfonic acid, sulfonic alkyl ester, thiosulfate, or sulfonamido; and
Y is cyano, isocyano, amino, alkyl amino, aminocarbonyl, aminocarbonyl alkyl, alkylcarbonylamino, amidino, guanidine, hydrazino, hydrazide, hydroxyl, alkoxy, aryloxy, hetroaryloxy, cyloalkyloxy, carbonyloxy, alkylcarbonyloxy, haloakylcarbonyloxy, arylcarbonyloxy, carbonylperoxy, alkylcarbonylperoxy, arylcarbonylperoxy, phosphate, alkylphosphate esters, sulfonic acid, sulfonic alkyl ester, thiosulfate, thiosulfenyl, sulfonamide, —R²³R²⁴, wherein R²³ is S, SS, Po, Po—Po, Se, Se—Se, Te, or Te—Te, and R²⁴ is defined as for R²¹, or Y is 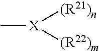
wherein X, R²¹ and R²², are as defined herein.

27. The method of claim 24, wherein said chalcogenide is selected from hydrogen sulfide (H₂S), sodium sulfide (Na₂S), sodium hydrogen sulfide (NaHS), potassium sulfide (K₂S), potassium hydrogen sulfide (KHS), lithium sulfide (Li₂S), rubidium sulfide (Rb₂S), cesium sulfide (Cs₂S), ammonium sulfide ((NH₄)₂S), ammonium hydrogen sulfide (NH₄)HS, beryllium sulfide (BeS), magnesium sulfide (MgS), calcium sulfide (CaS), strontium sulfide (SrS), barium sulfide (BaS), hydrogen selenide (H₂Se), and hydrogen telluride (H₂Te).

28. The method of claim 24, wherein said chalcogenide is administered intravenously, intradermally, intraarterially, intraperitoneally, intralesionally, intracranially, intraarticularly, intraprostaticaly, intrapleurally, intratracheally, intranasally, intrathecally, intravitreally, intravaginally, intrarectally, topically, intratumorally, intramuscularly, intraperitoneally, intraocularly, subcutaneously, subconjunctival, intravesicularlly, mucosally, intrapericardially, intraumbilically, intraocularally, orally, topically, locally, by inhalation, by injection, by infusion, by continuous infusion, by localized perfusion, via a catheter, or via a lavage.

29. The method of claim 24, wherein said chalcogenide is provided as a gas, semi-solid liquid, liquid, or solid.

30. The method of claim 23, wherein said stasis-inducing treatment comprises exposure of the patient in need thereof to a low oxygen environment.

31. The method of claim 30, wherein said patient in need thereof is exposed to an oxygen environment comprising less than about 1% O₂, less than about 0.1% O₂, less than about 0.01% O₂, or about 0.001% O₂.

32-34. (canceled)

35. The method of claim 23, wherein said stasis-inducing treatment comprises exposure of the patient in need thereof to a treatment regimen which lowers the core temperature of said patient.

36. The method of claim 35, wherein the core body temperature of the patient is reduced by at least about 5° F., by at least about 10° F., or by at least about 20° F.

37. (canceled)

38. (canceled)

39. The method of claim 23, wherein a portion of blood from said patient is withdrawn, such that blood flow to said cancer is reduced or eliminated.

40. The method of claim 23, wherein a portion of blood in said patient is diverted away from said cancer, such that blood flow to said cancer is reduced or eliminated.

41. The method of claim 23, wherein said dichloroacetate or salts thereof or chemical equivalents thereof is administered at a dose of at least about 10 mg/kg/day, at least about 25 mg/kg/day, or at least about 50 mg/kg/day.

42. (canceled)

43. (canceled)

44. The method of claim 23, wherein said dichloroacetate or salts thereof or chemical equivalents thereof is administered orally.

45. The method of claim 23, wherein said dichloroacetate or salts thereof or chemical equivalents thereof is administered in the form of a pharmaceutical composition comprising dichloroacetate or salts thereof or chemical equivalents thereof and a pharmaceutically acceptable carrier.

46. The method of claim 23, wherein said dichloroacetate or salts thereof or chemical equivalents thereof is administered before, during, and/or after the patient is subjected to a stasis-inducing treatment.

47. The method of claim 23, wherein said cancer is prostate cancer, bladder cancer, renal cancer, uterine cancer, ovarian cancer, testicular cancer, rectal cancer, colon cancer, lung cancer, breast cancer, pancreatic cancer, melanoma and other skin cancers, stomach cancer, brain cancer, liver cancer, adrenal cancer, kidney cancer, thyroid cancer, basal cell carcinoma, squamous cell carcinoma of both ulcerating and papillary type, metastatic skin carcinoma, medullary carcinoma, osteo sarcoma, Ewing's sarcoma, veticulum cell sarcoma, Kaposi's sarcoma, neuroblastoma, retinoblastoma, cutaneous T-cell lymphoma (CTCL), noncutaneous peripheral T-cell lymphoma, lymphoma associated with human T-cell lymphotrophic virus (HTLV), adult T-cell leukemia/lymphoma (ATLL), acute lymphocytic leukemia, acute nonlymphocytic leukemia, chronic lymphocytic leukemia, chronic myelogenous leukemia, Hodgkin's disease, non-Hodgkin's lymphoma, and multiple myeloma.
